(19) United States
(12) Patent Application Publication
(10) Pub. No.: US 2015/0201842 A1
(43) Pub. Date: Jul. 23, 2015

(54) MEASUREMENT PROBE AND OPTICAL MEASUREMENT SYSTEM

(71) Applicant: OLYMPUS MEDICAL SYSTEMS CORP., Tokyo (JP)

(72) Inventors: Seiki TORIYAMA, Tokyo (JP); Yoshimine KOBAYASHI, Tokyo (JP); Kazuhiro GONO, Sagamihara-shi (JP); Kenji KAMIMURA, Nagaoka-shi (JP)

(21) Appl. No.: 14/597,651

(22) Filed: Jan. 15, 2015

Related U.S. Application Data
(60) Provisional application No. 61/929,710, filed on Jan. 21, 2014.

(51) Int. Cl. A61B 5/00; G01N 21/49; G01N 21/47

(52) U.S. Cl. CPC A61B 5/0084; G01N 21/4738; G01N 21/49; A61B 5/0075

(57) ABSTRACT

A measurement probe includes a fiber configured to propagate light to irradiate a target to be measured and receive scattered light returned from the target to be measured, an immersion adjustment mechanism configured to communicate outside of the measurement probe with inside of the measurement probe and adjust entrance of liquid from the outside into the measurement probe, and an alteration mechanism configured to alter the fiber by the liquid that has entered into the measurement probe.

### CROSS-REFERENCE TO RELATED APPLICATIONS

This application is based upon and claims the benefit of priority from U.S. Provisional Patent Application No. 61/929,710, filed on Jan. 21, 2014, the entire contents of which are incorporated herein by reference.

### BACKGROUND

1. Technical Field

The disclosure relates to a measurement probe and an optical measurement system for measuring optical characteristics of body tissue.

2. Related Art

In recent years, optical measurement apparatuses are known, which irradiate body tissue with illumination light, and detect, based on measurement values of detected light reflected or scattered from the body tissue, characteristics (properties) of the body tissue. An optical measurement apparatus is used in combination with an endoscope for observing an organ such as a digestive organ. As such an optical measurement apparatus, an optical measurement apparatus is proposed (see Japanese Patent No. 5049415), which uses low-coherence enhanced backscattering (LEBS) for detecting characteristics of body tissue by irradiating low-coherence white light having a short spatial coherence length from an illumination fiber of a measurement probe to body tissue, detecting scattered light incident at mutually different angles using a plurality of light receiving fibers, and measuring an intensity distribution of the scattered light using spectrometers provided for the respective light receiving fibers.

Because the above mentioned measurement probe is inserted into a body, one that is sterilized for each examination always needs to be used. In order to maintain cleanliness for each examination, for the optical measurement apparatus, for example: a measurement probe that becomes reusable by subjecting the measurement probe that has been used to a cleaning treatment (a treatment using a cleaning liquid or a disinfection liquid or a steam sterilization treatment using an autoclave or the like); or a disposable measurement probe that is supposed to be discarded once used, is used.

### SUMMARY

In some embodiments, a measurement probe includes a fiber configured to propagate light to irradiate a target to be measured and receive scattered light returned from the target to be measured, an immersion adjustment mechanism configured to communicate outside of the measurement probe with inside of the measurement probe and adjust entrance of liquid from the outside into the measurement probe, and an alteration mechanism configured to alter the fiber by the liquid that has entered into the measurement probe.

In some embodiments, an optical measurement system includes the above-described measurement probe; and an optical measurement apparatus to which the measurement probe is detachably connected and which is configured to supply illumination light to the measurement probe, receive the scattered light emitted from the measurement probe; and measure optical characteristics of the target to be measured.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a perspective diagram schematically illustrating a configuration of an optical measurement system according to a first embodiment of the present invention;

FIG. 2 is a block diagram schematically illustrating the configuration of the optical measurement system according to the first embodiment of the present invention;

FIG. 3 is a partial cross section diagram schematically illustrating a configuration of main parts of a measurement probe according to the first embodiment of the present invention;

FIG. 4 is a partial cross section diagram schematically illustrating a configuration of main parts of the measurement probe according to the first embodiment of the present invention;

FIG. 5 is a perspective diagram schematically illustrating a configuration of main parts of the measurement probe according to the first embodiment of the present invention;

FIG. 6 is a cross section diagram along line A-A illustrated in FIG. 4;

FIG. 7 is a cross section diagram along line A-A illustrated in FIG. 4 and illustrating a state in which water has entered into the measurement probe;

FIG. 8 is a diagram illustrating a situation in which the optical measurement system according to the first embodiment of the present invention is used in an endoscopic system;

FIG. 9 is a perspective diagram schematically illustrating a configuration of main parts of a measurement probe according to a modified example 1-1 of the first embodiment of the present invention;

FIG. 10 is a partial cross section diagram schematically illustrating a configuration of main parts of the measurement probe according to the modified example 1-1 of the first embodiment of the present invention;

FIG. 11 is a schematic diagram illustrating a configuration of main parts of a measurement probe according to a modified example 1-2 of the first embodiment of the present invention;

FIG. 12 is a partial cross section diagram schematically illustrating a configuration of main parts of a measurement probe according to a second embodiment of the present invention;

FIG. 13 is a cross section diagram along line B-B illustrated in FIG. 12; and

FIG. 14 is a cross section diagram along line B-B illustrated in FIG. 12 and illustrating a state in which water has entered into the measurement probe.

### DETAILED DESCRIPTION

Hereinafter, with reference to the drawings, preferable embodiments of a measurement probe and an optical measurement system according to the present invention will be described in detail. The present invention is not limited by the embodiments. Further, in describing the drawings, the same portions are appended with the same reference signs. Further, the drawings are schematic, and it is to be noted that the relation between the thickness and width of each component and the ratios among the respective components are different from the actual. Further, a portion is included, which has different size relations and ratios among the drawings.

### First Embodiment

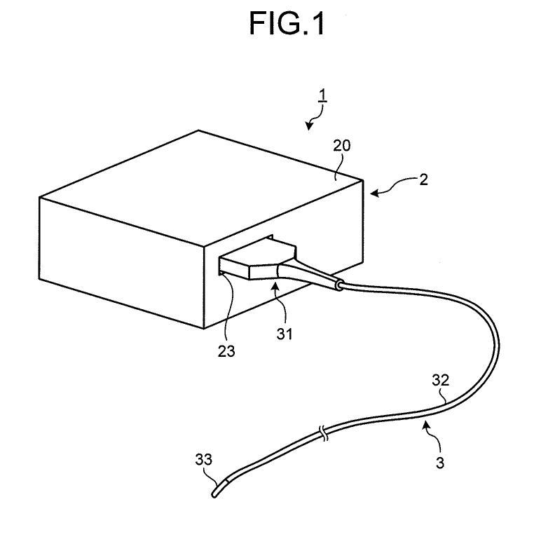

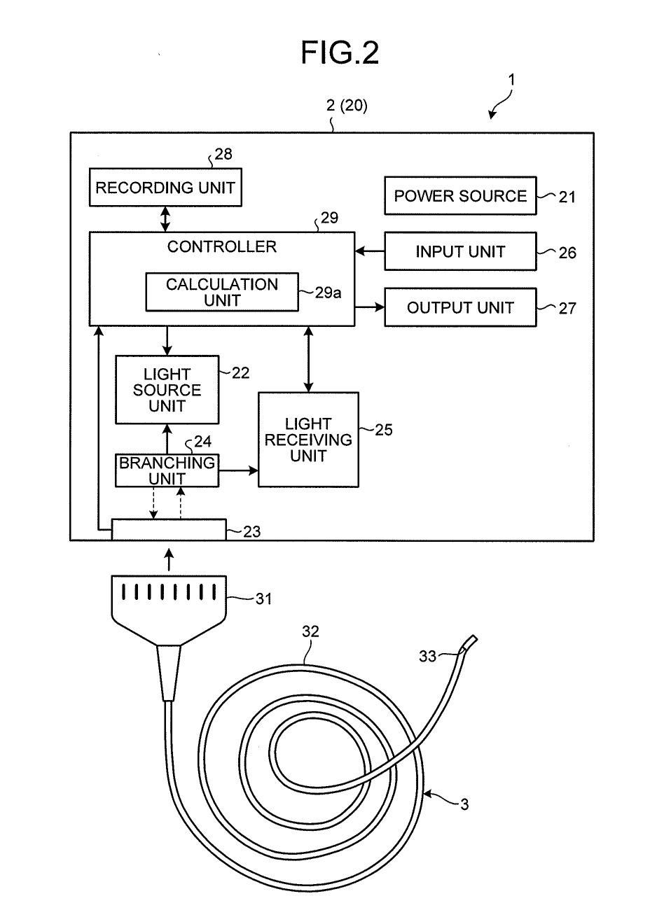

FIG. 1 is a perspective diagram schematically illustrating a configuration of an optical measurement system according to a first embodiment of the present invention. FIG. 2 is a block diagram schematically illustrating the configuration of the optical measurement system according to the first embodiment. An optical measurement system 1 illustrated in FIG. 1 includes: an optical measurement apparatus 2 that performs measurement of optical characteristics with respect to a target to be measured such as body tissue, which is a scatterer, to detect characteristics (properties) of the target to be measured; and a measurement probe 3 that is detachable from the optical measurement apparatus 2, is inserted into a subject, and is for measurement. In this specification, the measurement probe 3 is described as that of a disposable type, which is supposed to be discarded once used.

First, the optical measurement apparatus 2 is described. The optical measurement apparatus 2 includes a main body unit 20. In the main body unit 20, a power source 21, a light source unit 22, a connection unit 23, a branching unit 24, a light receiving unit 25, an input unit 26, an output unit 27, a recording unit 28, and a controller 29 are provided. The power source 21 supplies electric power to each structural element of the optical measurement apparatus 2.

The light source unit 22 is realized by using an incoherent light source such as a white light emitting diode (LED), a xenon lamp, a tungsten lamp, and a halogen lamp, and as necessary, one or more lenses, for example, a condenser lens, a collimator lens, or the like. The light source unit 22 outputs to the measurement probe 3 via the connection unit 23, incoherent light having at least one spectral component for irradiating the target to be measured.

The connection unit 23 detachably connects a connector portion 31 of the measurement probe 3 to the optical measurement apparatus 2. The connection unit 23 outputs light emitted by the light source unit 22 to the measurement probe 3, and outputs scattered light, which is emitted from the measurement probe 3 and returned from the target to be measured, to the light receiving unit 25. The connection unit 23 outputs, to the controller 29, information related to presence or absence of connection of the measurement probe 3.

The branching unit 24 condenses or bends the light from the light source unit 22 to be guided to the measurement probe 3 and guides light from an object to be examined via the measurement probe 3 to the light receiving unit 25. The branching unit 24 includes an optical system that is realized by using one or more lenses, for example, a condenser lens, a collimator lens, or the like.

The light receiving unit 25 receives and measures scattered light, which is illumination light emitted from the measurement probe 3 and returned from the target to be measured. The light receiving unit 25 is realized by using a plurality of spectrometers, light receiving sensors, or the like. Specifically, the light receiving unit 25 is provided with the spectrometers, according to the number of later described light receiving fibers of the measurement probe 3. The light receiving unit 25 measures a spectral component and an intensity distribution of scattered light incident from the measurement probe 3 and performs measurement of each wavelength. The light receiving unit 25 outputs results of the measurement to the controller 29.

The input unit 26 is realized by using push-type switches, a touch panel, or the like, and receives input of an instruction signal instructing activation of the optical measurement apparatus 2 or instruction signals instructing various other operations and outputs it to the controller 29.

The output unit 27 is realized by using a display of a liquid crystal or electroluminescence, a speaker, and the like, and outputs information related to various processes in the optical measurement apparatus 2. Further, the output unit 27 displays on a display, under control by the controller 29, numerical values such as intensities of light received by the light receiving unit 25 (characteristic values calculated by a calculation unit 29a described later).

The recording unit 28 is realized by using a volatile memory or a non-volatile memory, and records therein various programs for operating the optical measurement apparatus 2, and various data and various parameters used in an optical measurement process. The recording unit 28 temporarily records therein information being processed by the optical measurement apparatus 2. Further, the recording unit 28 records therein results of the measurement by the optical measurement apparatus 2 in association with the subject to be measured. The recording unit 28 may be configured by using a memory card or the like inserted from outside of the optical measurement apparatus 2.

The controller 29 is configured by using a central processing unit (CPU) or the like. The controller 29 controls operations of a process by each unit of the optical measurement apparatus 2. The controller 29 controls the operations of the optical measurement apparatus 2 by performing transfer or the like of instruction information and data corresponding to each unit of the optical measurement apparatus 2. The controller 29 records the results of the measurement by the light receiving unit 25 in the recording unit 28. The controller 29 has the calculation unit 29a.

Based on the results of the measurement by the light receiving unit 25, the calculation unit 29a performs a plurality of calculation processes and calculates characteristic values related to the characteristics of the target to be measured. The types of these characteristic values are set according to the instruction signal received by the input unit 26, for example.

Next, the measurement probe 3 is described. The measurement probe 3 includes: the connector portion 31 detachably connected to the connection unit 23 of the optical measurement apparatus 2; a flexible portion 32 having a flexibility; and a distal end portion 33, which emits the illumination light supplied from the light source unit 22 and receives the scattered light from the target to be measured. The measurement probe 3 connects to the optical measurement apparatus 2 at one end thereof, propagates light from the light source unit 22 to irradiate the target to be measured, contacts the target to be measured at another end thereof, and receives the scattered light (returned light), which is obtained by irradiating the target to be measured, propagating inside the target to be measured, and returning.

Inside the measurement probe 3, a fiber is arranged. Specifically, the measurement probe 3 includes a fiber (later described fiber 300), which propagates the light from the light source unit 22 and irradiates the target to be measured with the illumination light, the fiber on which the scattered light (returned light) returning from the target to be measured is incident at different angles. The fiber is configured by using, for example, a step index single core fiber or a multi-mode fiber. The scattered light includes light scattered from the target to be measured, as well as light reflected from the target to be measured.

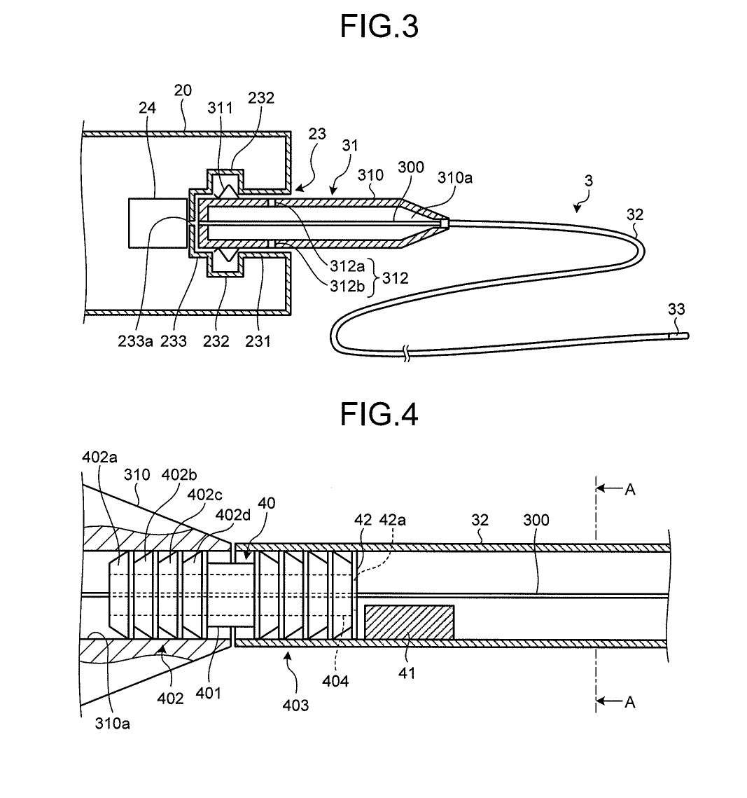

FIG. 3 is a partial cross section diagram schematically illustrating a configuration of main parts (near the connection unit 23) of the measurement probe according to the first embodiment. FIG. 3 is a cross section diagram for a cross section cut along a plane parallel to a paper surface. The connection unit 23 has, formed therein: a first hole 231 that forms an opening on a lateral side of the main body unit 20; a second hole 232 that communicates with the first hole 231 and has a diameter greater than a diameter of the first hole 231; and a concave portion 233 that communicates with the second hole 232. The first hole 231 forms a hollow space that is able to house the connector portion 31. In the concave portion 233, a hole portion 233a is formed on a bottom portion of a concave shape thereof. The branching unit 24 emits illumination light to the outside or receives the returned light, via the hole portion 233a.

The connector portion 31 includes: a main body portion 310; and a plate spring 311 that is arranged at an end portion of the main body portion 310 at a side to be connected to the connection unit 23, arranged along a lateral side of the main body portion 310, and bent so that a part thereof protrudes from the lateral side, and the connector portion 31 is connected to the connection unit 23 at one end thereof and is coupled to the flexible portion 32 at the other end thereof.

In the main body portion 310, a hollow portion 310a that forms a hollow space in the main body portion 310 is formed. The fiber 300 is inserted through the hollow portion 310a.

The plate spring 311 has a shape memory property. By this shape memory property, upon connection of the connector portion 31 to the connection unit 23, after being pressed against the first hole 231 and elastically deformed, a shape thereof is recovered in the second hole 232. When the connector portion 31 is connected to the connection unit 23, the plate spring 311 and a step portion formed by the first hole 231 and the second hole 232 are in a locked state, and thus the connector portion 31 is able to be restricted from being separated from the connection unit 23. Other than the plate spring 311, a known locking member may be used, such as an O-ring that is elastically deformable or a pin that is provided to freely move back and forth from an outer surface of the main body portion 310.

The main body portion 310 has a hole portion 312 that communicates inside of the main body portion 310 with outside of the main body portion 310. The hole portion 312 includes a first hole portion 312a that is provided on a lateral side of the main body portion 310, and a second hole portion 312b that is provided on a lateral side of the main body portion 310, the lateral side being on an opposite side of the first hole portion 312a. The hole portion 312 is provided at a position such that the hole portion 312 is positioned inside the connection unit 23 (the first hole 231, the second hole 232, and the concave portion 233) when the connector portion 31 is connected to the connection unit 23. The hole portion 312 may be made of a single hole or three or more holes, as long as the hole portion 312 is able to exhaust a gas inside the main body portion 310 to outside thereof.

FIG. 4 is a partial cross section diagram schematically illustrating a configuration of main parts (a connection part between the connector portion 31 and the flexible portion 32) of the measurement probe according to the first embodiment. The connector portion 31 and the flexible portion 32 are coupled by a connecting member 40. Further, inside the flexible portion 32, an alteration mechanism is provided, which is formed of: an expansive body 41 that is provided at a distal end side of the connecting member 40 and expands by absorbing moisture; and a cutting member 42 that is provided between the connecting member 40 and the expansive body 41 and cuts the fiber 300.

The connecting member 40 is formed by using an elastically deformable material. The connecting member 40 includes: a base portion 401 that is approximately cylindrical; a first connecting portion 402 that extends from one end of the base portion 401 along a longitudinal direction thereof and is pressed into the hollow portion 310a of the main body portion 310; and a second connecting portion 403 that extends to another end of the base portion 401 along the longitudinal direction and pressed into the flexible portion 32. In each of the first connecting portion 402 and the second connecting portion 403, a hollow space communicating with a hollow space of the base portion 401 is formed. Thereby, a through hole 404 penetrating in a longitudinal direction is formed in the connecting member 40. The fiber 300 is inserted into the through hole 404.

In the first connecting portion 402, a plurality of spindle shaped bodies 402a to 402d, which are approximately spindle shaped, and in which a hollow space is formed along central axes thereof, are arranged in order with their central axes being aligned with one another. Therefore, a side view seen from a direction orthogonal to the central axes forms a serrated shape. The maximum diameters of the spindle shaped bodies 402a to 402d, among their diameters in the direction orthogonal to their central axes, are larger than a diameter of the hollow portion 310a. The maximum diameters of the spindle shaped bodies 402a to 402d may be different from one another and shapes thereof may be different from one another, as long as the above condition on the diameters are satisfied.

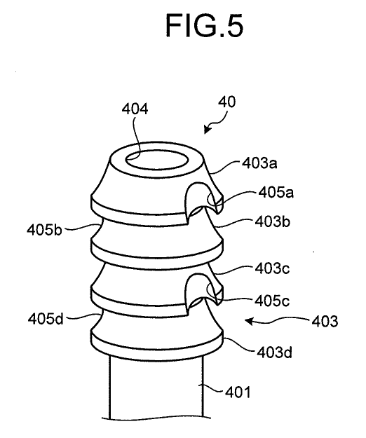

FIG. 5 is a perspective diagram schematically illustrating a configuration of main parts (second connecting portion 403) of the measurement probe according to the first embodiment. In the second connecting portion 403, a plurality of spindle shaped bodies 403a to 403d, which are approximately spindle shaped, and in which a hollow space is formed along their central axes, are arranged in order with their central axes aligned with one another. Therefore, a side view seen from a direction orthogonal to the central axes forms a serrated shape. The maximum diameters of the spindle shaped bodies 403a to 403d, among their diameters in a direction orthogonal to their central axes, are greater than an inner diameter of the flexible portion 32. The maximum diameters of the spindle shaped bodies 403a to 403d may be different from one another and shapes thereof may be different from one another, as long as the above condition on the diameters are satisfied.

Further, in the spindle shaped bodies 403a to 403d, notched portions 405a to 405d, which are formed by portions of their lateral sides being notched off, are respectively formed. The notched portions 405a to 405d are respectively provided on opposite lateral sides in adjacent ones of the spindle shaped bodies 403a to 403d. Specifically, in the spindle shaped body 403a and spindle shaped body 403c, the notched portion 405a and notched portion 405c are provided along the central axes. In the spindle shaped body 403b and spindle shaped body 403d, the notched portion 405b and notched portion 405d are provided on an opposite side of the notched portion 405a and notched portion 405c with respect to the central axes.

When the second connecting portion 403 is pressed into the flexible portion 32, the spindle shaped bodies 403a to 403d come into a state of being pressed against an inner wall of the flexible portion 32. In this state, the spindle shaped bodies 403a to 403d form hollow spaces, together with the inner wall of the flexible portion 32. The hollow spaces formed by the spindle shaped bodies 403a to 403d and the flexible portion 32 are communicated with one another by the notched portions 405a to 405c. Further, the hollow space formed by the spindle shaped body 403d and the inner wall of the flexible portion 32 is communicated by the notched portion 405d with a hollow space formed of the base portion 401 and the inside of the flexible portion 32.

Because the notched portions 405a to 405d are provided at positions where adjacent ones of the notched portions are opposite to each other, the hollow space formed by the spindle shaped bodies 403a to 403d and the inner wall of the flexible portion, that is, a path of the hollow space communicated by the connecting member 40 and joining the inside and outside of the flexible portion is zigzagged. In other words, this path (immersion adjustment mechanism) extends in non-parallel with a longitudinal direction of the flexible portion 32 (measurement probe 3).

The expansive body 41 has a water absorbing property and is formed by using a material that increases in volume by water absorption.

The cutting member 42 forms a ring shape having an outer diameter that is equivalent to the inner diameter of the flexible portion 32 or smaller than the inner diameter. An inner diameter of the cutting member 42 is smaller than a diameter of the through hole 404. On an end face at an inner diameter side of the cutting member 42, a blade portion 42a that is sharp in shape is formed. The fiber 300 is cut by coming into contact with the blade portion 42a.

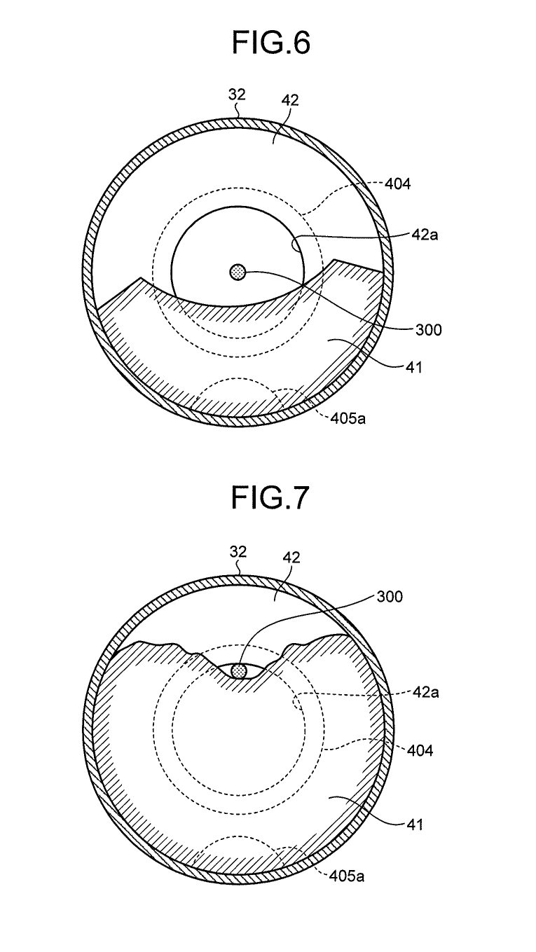

FIG. 6 is a cross section diagram along line A-A illustrated in FIG. 4. If the measurement probe 3 having the above described configuration is immersed in water, the water gets in from a gap between the connector portion 31 and the flexible portion 32. When this happens, at a connector portion 31 side, at least the spindle shaped bodies 402a to 402d are pressed against the hollow portion 310a, and the water does not enter the hollow portion 310a. Since at a flexible portion 32 side, the notched portions 405a to 405d are formed in the spindle shaped bodies 403a to 403d and the inside and outside of the flexible portion 32 are communicated with each other by the above described path, the water enters inside the flexible portion 32 from the notched portion 405a.

FIG. 7 is a cross section diagram along line A-A illustrated in FIG. 4 and illustrating a state in which the water has entered into the measurement probe. If the water enters inside the flexible portion 32, the expansive body 41 absorbs the water and expands. By the expansion of the expansive body 41, the fiber 300 is pushed up to come into contact with the blade portion 42a. Thereafter, by the expansive body 41 expanding further, the fiber 300 is pressed against the blade portion 42a to be cut.

As described above, by having the above described configuration as the disposable measurement probe 3, when cleaned by a cleaning liquid, or subjected to a steam sterilization treatment with an autoclave, water enters inside the flexible portion 32 and through expansion of the expansive body 41, the fiber 300 is cut by the blade portion 42a. By alteration of the fiber 300 by this cut, even if the measurement probe 3 is attempted to be used after the cleaning process or the steam sterilization treatment, illumination light is not emittable therefrom, and thus measurement is not possible.

If the expansive body 41 is not expanded, the fiber 300 and the blade portion 42a are not pressed against each other and because a self-weight of the fiber 300 is extremely light, the fiber 300 is not cut just by contacting the blade portion 42a. Therefore, the fiber 300 is cut by being pressed against the blade portion 42a through the expansion of the expansive body 41.

Further, if water enters inside the flexible portion 32, a gas present inside the flexible portion 32 is exhausted to outside of the measurement probe 3 via the hole portion 312, and thus water is not restricted from entering inside the flexible portion by an increase in an internal pressure of the flexible portion 32. In other words, as the water enters inside the flexible portion 32, the gas inside thereof is exhausted to the outside of the measurement probe 3 via the hole portion 312.

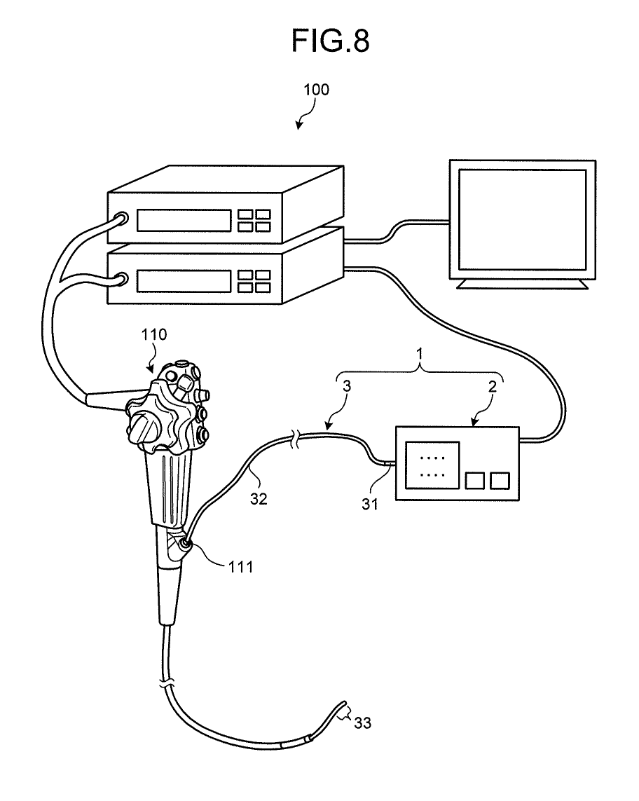

FIG. 8 is a diagram illustrating a situation in which the optical measurement system according to the first embodiment is used in an endoscopic system. In the above described optical measurement system 1, as illustrated in FIG. 8, the measurement probe 3 is inserted in the subject via a treatment tool channel 111 provided in an endoscopic apparatus 110 (endoscope) of an endoscopic system 100, an illumination fiber irradiates the target to be measured with the illumination light, and the plurality of light receiving fibers respectively receive the returned light of the illumination light reflected and/or scattered from the target to be measured at different scattering angles and propagate it to the light receiving unit 25 of the optical measurement apparatus 2. Thereafter, the calculation unit 29a calculates the characteristic values of the characteristics of the target to be measured, based on the results of the measurement by the light receiving unit 25.

According to the first embodiment of the present invention, since the flow path for water to come towards the flexible portion 32 is formed in the connecting member 40 that couples the connector portion 31 and the flexible portion 32 is formed, and the expansive body 41 that expands by water absorption and the cutting member 42 that cuts the fiber 300 are provided, to cut the fiber 300 by the entrance of water inside the flexible portion 32, reuse of a disposable measurement probe is preventable.

Further, according to the first embodiment, since the path of the hollow spaces communicated by the connecting member 40 and joining the inside and outside of the flexible portion is zigzagged, the path becomes longer than a straight lined path extending along a longitudinal direction of the connecting member 40. Therefore, an amount of water required to reach the expansive body 41 is greater than that for the straight-lined path, and thus, even if a very small amount of moisture generated during measurement enters, for example, even if moisture or the like adhered on hands of a user enters, the water is prevented from reaching the expansive body 41, and it thus becomes possible to suppress expansion of the expansive body 41, and to restrict the fiber 300 from being cut, just by such unexpectedly occurring entrance of water. The path through which a liquid comes may be formed straight-lined, as long as a length of the second connecting portion 403 in the longitudinal direction is of a length that is able to restrict the fiber 300 from being cut.

Further, according to the first embodiment, because the hole portion 312 is provided at the position that is positioned inside the connection unit 23 (the first hole 231, second hole 232, and concave portion 233) when the connector portion 31 is connected to the connection unit 23, even if water is spattered unexpectedly on the measurement probe 3, that water does not enter from this hole portion 312, and safety of the measurement probe 3 is able to be maintained.

### MODIFIED EXAMPLE 1-1

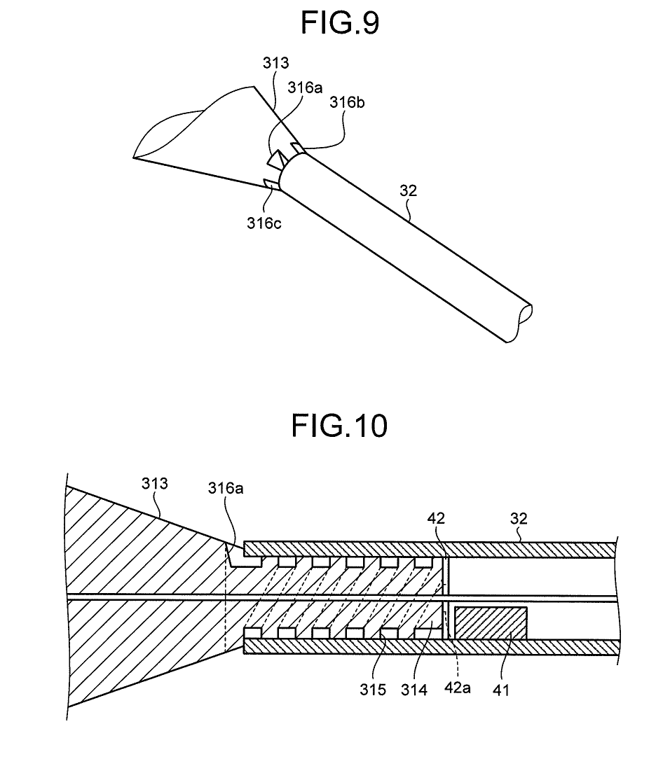

FIG. 9 is a perspective diagram schematically illustrating a configuration of main parts (a connection part between the connector portion 31 and the flexible portion 32) of a measurement probe according to a modified example 1-1 of the first embodiment. FIG. 10 is a partial cross section diagram schematically illustrating the configuration of the main parts (the connection part between the connector portion 31 and the flexible portion 32) of the measurement probe according to the modified example 1-1 of the first embodiment. In the modified example 1-1, a main body portion 313 is included in stead of the main body portion 310 of the connector portion 31 of the above described first embodiment. At a distal end of the main body portion 313 on the side where the flexible portion 32 is connected, a press-in portion 314 to be pressed into the flexible portion 32 is provided.

On an outer periphery of the press-in portion 314, a groove portion 315 is spirally formed from a distal end thereof. At a connection between the press-in portion 314 and the main body portion 313, hole portions 316a to 316c extending from the groove portion 315 are formed.

Accordingly, if cleaning with a cleaning liquid is performed or a steam sterilization treatment using an autoclave is performed, water enters the groove portion 315 via the hole portions 316a to 316c to allow the expansive body 41 to be expanded. Further, by forming the groove portion 315 spirally, a path for the water to reach the expansive body 41 is able to be made long and the expansive body 41 is able to be restricted from expanding just by entrance of water to an extent caused unexpectedly.

According to the description of the modified example 1-1, the hole portions 316a to 316c are formed, but as long as a liquid is able to enter the flexible portion 32, one or more hole portions may be provided.

### MODIFIED EXAMPLE 1-2

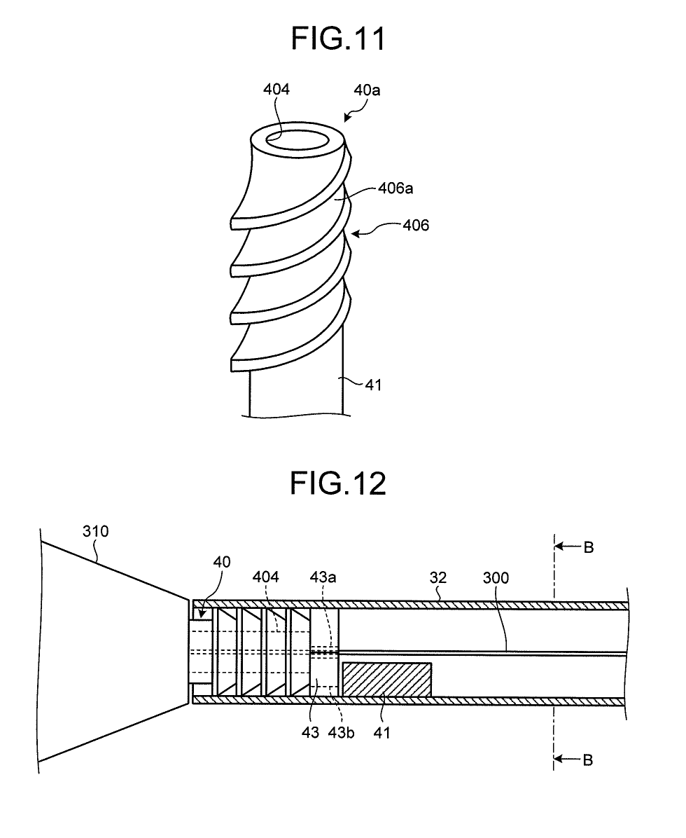

FIG. 11 is a schematic diagram illustrating a configuration of main parts (a connecting member) of a measurement probe according to a modified example 1-2 of the first embodiment. The second connecting portion 403 of the connecting member 40 according to the above described first embodiment is provided with the notched portions 405a to 405d in the spindle shaped bodies 403a to 403d to form the zigzagged path, but even by a second connecting portion 406 in which a groove portion 406a that is spiral like a connecting member 40a according to the modified example 1-2, the above described effects are obtainable.

### Second Embodiment

Next, a second embodiment of the present invention will be described. FIG. 12 is a partial cross section diagram schematically illustrating a configuration of main parts (a connection part between the connector portion 31 and the flexible portion 32) of a measurement probe according to the second embodiment. Structural elements, which are the same as those described above, are appended with the same reference signs. According to the description of the above described first embodiment, the fiber 300 is cut by the cutting member 42 having the blade portion 42a, but according to the second embodiment, the fiber 300 is held by a holding member 43 and the fiber is cut by a pressure due to the expansion of the expansive body 41.

In the measurement probe according to the second embodiment, the connector portion 31 and the flexible portion 32 are coupled by the above described connecting member 40. Further, inside the flexible portion 32: the expansive body 41 that is provided at the distal end side of the connecting member 40 and expands by absorbing moisture; and the holding member 43 that is provided between the connecting member 40 and the expansive body 41 and holds the fiber 300, are provided.

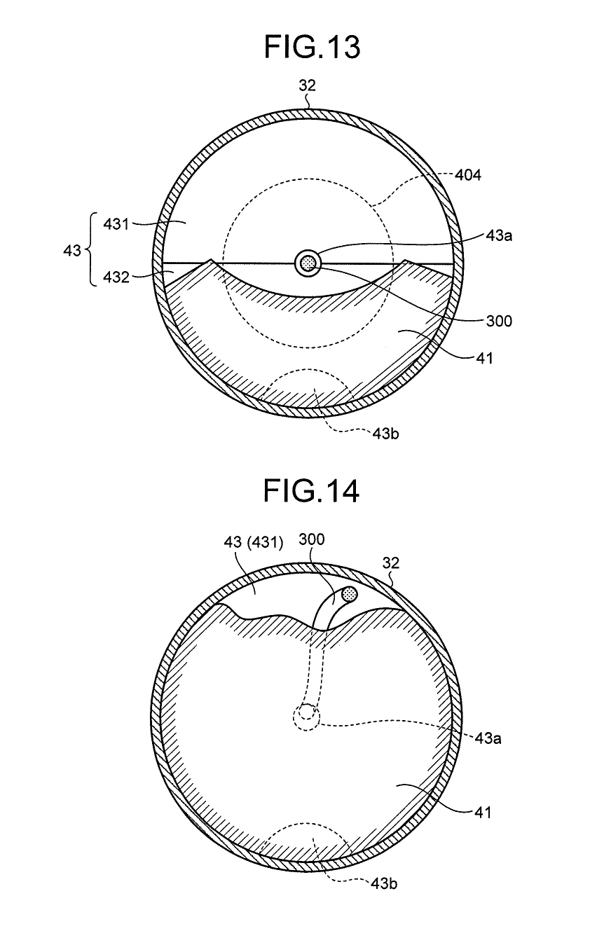

FIG. 13 is a cross section diagram along line B-B illustrated in FIG. 12. The holding member 43 has a cylindrical shape in which a through hole 43a is formed, which penetrates therethrough along a central axis thereof. A diameter of the through hole 43a is a little greater than a diameter of the fiber 300. Thereby, when the fiber 300 is in a state of being inserted in the through hole 43a, even if the flexible portion 32 is bent, the fiber 300 is able to follow the bend without placing a load on the fiber 300.

Further, on a lateral side of the holding member 43, a notched portion 43b, which is formed by performing notching along a central axis thereof, is provided. Thereby, the connecting member 40 is communicated with the expansive body 41.

The holding member 43 is formed, for example, by overlapping a first member 431 and a second member 432, which are approximately semicylinder, to face each other. The holding member 43 is formed of a hard resin or the like.

If the measurement probe having the above described configuration is immersed in water, the water enters from a gap between the connector portion 31 and the flexible portion 32. If the water enters from this gap, the water enters inside the flexible portion 32 through the above described path and the water that has entered therein reaches the expansive body 41 via the notched portion 43b.

FIG. 14 is a cross section diagram along line B-B illustrated in FIG. 12 and is a diagram illustrating a state in which water has entered into the probe. When the water that has entered inside the flexible portion 32 reaches the expansive body 41 via the notched portion 43b, the expansive body 41 absorbs the water and expands. By the expansion of the expansive body 41, the fiber 300 is pushed up to press the fiber 300 between the expansive body 41 and the holding member 43. Thereafter, by further expansion of the expansive body 41, the fiber 300 is broken.

As described, if cleaning with a cleaning liquid is performed or a steam sterilization treatment with an autoclave is performed, by entrance of water inside the flexible portion 32, the expansive body 41 expands, the fiber 300 is pressed between the expansive body 41 and the holding member 43, and thereby the fiber 300 is broken. By alteration of the fiber 300 due to this breakage, a transmissivity of the fiber 300 is significantly reduced. Accordingly, even if the measurement probe 3 is attempted to be used after the cleaning process or the steam sterilization process, since illumination light emitted from the fiber 300 is significantly reduced, measurement is not able to be performed.

According to the above described second embodiment of the present invention, because by forming the flow path for the water to come towards the flexible portion 32 in the connecting member 40 that couples the connector portion 31 and the flexible portion 32, and providing the expansive body 41 that expands by water absorption and the holding member 43 that holds the fiber 300, the fiber 300 is caused to be broken by the entrance of the water inside the flexible portion 32, reuse of a disposable measurement probe is preventable.

Further, according to the second embodiment, since the holding member 43 is formed by overlapping the approximately semicylindrical first member 431 and second member 432 to face each other, assembly of the holding member 43 and assembly of the measurement probe 3 are able to be readily performed.

According to the description of the above first and second embodiments, although the fiber 300 is cut by the alteration mechanism formed of one expansive body 41, and the cutting member 42 or holding member 43, two expansive bodies 41 may be arranged alternately to interpose the fiber therebetween by expansion.

Further, according to the description of the above first and second embodiments, the fiber 300 is cut by the alteration mechanism formed of the expansive body 41, and the cutting member 42 or holding member 43, but not being limited thereto, for example, a fiber may be used, which is reduced in its transmissivity (optical characteristics) by alteration (being cracked or dissolved) in an exposed portion upon exposure to a cleaning liquid or a disinfection liquid, to make remeasurement after cleaning impossible. In this case, the alteration mechanism corresponds to the fiber itself, which is formed using a material that alters at the portion exposed to the cleaning liquid or the disinfection liquid. Further, a material forming the connector portion 31 may be one that alters (is cracked or dissolved) upon exposure to a cleaning liquid, for example, so that attachment to the optical measurement apparatus 2 becomes impossible after the alteration.

Further, whether or not the measurement probe is usable may be allowed to be checked by holding in the connector portion 31, a liquid or a viscous liquid that reacts and changes in color upon exposure to water, a cleaning liquid, or the like, for example, and checking the color change by the reaction of this liquid or viscous liquid caused by the exposure or impact due to the cleaning.

According to some embodiments, it is possible to prevent reuse of a disposable measurement probe.

The present invention may include various embodiments not described herein, and various design changes and the like may be made within the scope of the technical ideas defined by the claims.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A measurement probe, comprising:
a fiber configured to propagate light to irradiate a target to be measured and receive scattered light returned from the target to be measured;
an immersion adjustment mechanism configured to communicate outside of the measurement probe with inside of the measurement probe and adjust entrance of liquid from the outside into the measurement probe; and
an alteration mechanism configured to alter the fiber by the liquid that has entered into the measurement probe.

2. The measurement probe according to claim 1, wherein the immersion adjustment mechanism is a flow path that joins the outside of the measurement probe and the alteration mechanism and extends in non-parallel with a longitudinal direction of the measurement probe.

3. The measurement probe according to claim 1, wherein the alteration mechanism comprises:
an expansive body configured to absorb the liquid and expand; and
a cutting member that is provided between the immersion adjustment mechanism and the expansive body, and has a hole therein through which the fiber is configured to be inserted, a distal end face of the hole having a sharp shape.

4. The measurement probe according to claim 1, wherein the alteration mechanism comprises:
an expansive body configured to absorb the liquid and expand; and
a holding member that is provided between the immersion adjustment mechanism and the expansive body, and has a hole therein for holding the fiber.

5. An optical measurement system, comprising:
the measurement probe according to claim 1; and
an optical measurement apparatus to which the measurement probe is detachably connected and which is configured to supply illumination light to the measurement probe, receive the scattered light emitted from the measurement probe, and measure optical characteristics of the target to be measured.

6. The optical measurement system according to claim 5, wherein
the measurement probe has a connector portion configured to connect to the optical measurement apparatus,
the optical measurement apparatus has a connection unit configured to house a part of the connector portion to connect to the measurement probe, and
the connector portion has a hole portion therein for communicating inside of the connector portion with outside of the connector portion, at a position that is arranged inside the connection unit when the connection unit houses the part of the connector portion to connect to the measurement probe.
